(19) United States
(12) Patent Application Publication
(10) Pub. No.: US 2007/0232574 A1
(43) Pub. Date: Oct. 4, 2007

(54) 2-Oxothiazolidine 4-Carboxylic acid compounds for promoting desquamation of the skin

(75) Inventors: Jean-Baptiste Galey, Aulnay-Sous-Bois (FR); Dominique Bernard, Paris (FR); Lucie Simonetti, Paris (FR)

Correspondence Address: BUCHANAN, INGERSOLL & ROONEY PC, POST OFFICE BOX 1404, ALEXANDRIA, VA, 22313-1404, US

(73) Assignee: SOCIETE L'OREAL, S.A., Paris (FR)

(21) Appl. No.: 11/806,218

(22) Filed: May 30, 2007

Related U.S. Application Data
(62) Division of application No. 10/440,316, filed on May 19, 2003.
Application No. 10/440,316 is a continuation of application No. PCT/FR01/03523, filed on Nov. 12, 2001.

(30) Foreign Application Priority Data
Nov. 17, 2000 (FR) 00/14865

(51) Int. Cl. A61K 31/426; A61K 31/60; A61K 31/65; A61P 17/00

(52) U.S. Cl. 514/154; 514/161; 514/369

(57) ABSTRACT

2-oxothiazolidine-4-carboxylic acid compounds having the structural formula (I): 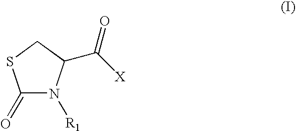 are suited for promoting desquamation of the skin and/or stimulating epidermal renewal and/or combating aging of the skin.

### CROSS-REFERENCE TO EARLIER APPLICATIONS

This application is a divisional of copending U.S. patent application Ser. No. 10/440,316, filed May 19, 2003, which is a continuation of International Application No. PCT/FR01/03523, filed Nov. 12, 2001 and designating the United States (published in the French language on May 23, 2002 as WO 02/39976 A1; the title and abstract of which were also published in English), and claiming the priority under 35 U.S.C. § 119 of FR-00/14865, filed Nov. 17, 2000, the earlier applications all incorporated by reference herein in their entireties and relied upon.

### BACKGROUND OF THE INVENTION

1. Technical Field of the Invention

The invention relates to the use, in a composition or for the manufacture of a composition, of at least one 2-oxothiazolidine-4-carboxylic acid derivative, the derivative or the composition being intended to promote desquamation of the skin and/or to stimulate epidermal renewal and thus to combat intrinsic aging of the skin.

The invention also relates to a nontherapeutic regime or regimen for treating the skin which is intended to promote desquamation and/or to stimulate epidermal renewal and thus to combat intrinsic aging of the skin, which comprises topically applying to the skin a composition comprising at least one 2-oxothiazolidine-4-carboxylic acid derivative.

2. Description of Related/Prior Art

Aging of the skin results from two distinct and independent processes involving intrinsic or extrinsic factors.

Intrinsic or chronobiological aging corresponds to “normal” or age-related physiological aging.

Extrinsic aging corresponds to aging caused generally by the environment and more particularly photoaging due to exposure to sunlight, to light or to any other radiation (EP-A2-0,815,840, Kligman, A. M. et al., Journal of Cutaneous Aging and Cosmetic Dermatology, Vol. 1, No. 1, pp. 5-12 (1988)).

The present invention relates only to intrinsic or physiological aging of the skin.

Aging of the skin is generally reflected by the appearance of wrinkles and fine lines, by yellowing of the skin which develops a wizened appearance accompanied by the appearance of pigmentation marks, by disorganization of the elastin and collagen fibers resulting in a loss of elasticity, suppleness and firmness, or by the appearance of telangiectasias.

The changes in the skin due to intrinsic aging are the consequence of a genetically programmed senescence involving endogenous factors. This intrinsic aging is especially reflected by a slowing-down in the renewal of the epidermal cells and the appearance of wrinkles or fine lines.

In contrast, extrinsic aging results, in the dermis, from the degradation of the collagen fibers, the consequence of which is especially clinical impairments such as heavy wrinkles and the formation of a flaccid and weather-beaten skin.

Desquamation is a natural phenomenon associated with the fact that the epidermis, which constitutes the upper layer of the skin, is in constant regeneration.

The human epidermis consists of several layers of cells in which mainly four types of cells are found: keratinocytes, which form the vast majority, melanocytes, Langerhans cells and Merkel cells. The distribution of these cells in several superposed layers explains the stratified nature of the epidermis.

The epidermis is conventionally divided into a basal layer of keratinocytes which constitutes the germinative layer of the epidermis, a “spiny” layer consisting of several layers of polyhedric cells arranged on the germinative cells, a “granulous” layer consisting of flattened cells containing distinct cytoplasmic inclusions, keratohyalin grains, and finally an upper layer known as the horny layer (or stratum corneum), consisting of keratinocytes at the final stage of their differentiation, known as corneocytes. The corneocytes are mummified anuclear cells which are derived from the keratinocytes and are removed by desquamation. This loss at the surface is compensated for by the migration of cells from the basal layer towards the surface of the epidermis. This constitutes a perpetual renewal of the epidermis. A forced removal of the horny layer accelerates the renewal and makes it possible to combat aging of the skin.

The corneocytes are mainly composed of a fibrous matrix containing cytokeratins, surrounded by a very strong structure 15 nm thick, known as the horny or cornified envelope. The stacking of these corneocytes constitutes the horny layer which is responsible for the barrier function of the epidermis. During the normal process of desquamation, the uppermost corneocytes become detached from the surface of the epidermis.

Intercellular structures derived from desmosomes, known as corneosomes or corneodesmosomes, have been described in the horny layer. Recent studies have shown their major importance in intercorneocytic cohesion and also in the desquamation process.

Corneodesmosine, which has been characterized elsewhere in EP-A-0,972,042 by the assignee hereof, is a protein of the horny layer of the epidermis which is involved in intercorneocytic cohesion and which is a constituent of the corneodesmosomes.

In the horny layer, a close correlation exists between cell dissociation and the proteolysis of certain corneodesmosomal components, for instance desmoglein I and corneodesmosine. Several serine proteases of trypsin or chymotrypsin type appear to be involved in the proteolysis of corneodesmosomes, such as, in particular, proteases of chymotrypsin-like or trypsin-like type (Lundström A., Egelrud T., The Journal of Investigative Dermatology; 1988, 91:340-343 and 1990, 84:216-220).

The prior art discloses various agents for combating aging of the skin, in particular by promoting desquamation, that is to say the removal of the “dead” cells at the surface of the horny layer of the epidermis. This “desquamating” property is also referred to, erroneously, as a keratolytic property.

Thus, U.S. Pat. No. 4,603,146 discloses the use of retinoic acid and its derivatives in cosmetic compositions for combating aging of the skin.

Moreover, many patents and publications (see for example EP-A-413,528) and also many commercial cosmetic compositions teach the use of α-hydroxy acids, for instance lactic acid, glycolic acid or citric acid, for treating aging of the skin.

Finally, β-hydroxy acids and more especially salicylic acid and derivatives thereof are known for their desquamating properties (see WO-A-93/10756 and U.S. Pat. No. 4,767,750).

The fact remains that the desire to conserve a youthful appearance always leads to the incessant search for novel compounds and/or novel compositions for maintaining or improving the appearance of the skin.

Certain cosmetic active agents are capable of stimulating the degradation of corneodesmosomal proteins and thus desquamation, undoubtedly, as has been seen previously, by promoting the activity of proteases involved in this process.

In this perspective, EP-A2-0,852,949 (Shiseido) has disclosed that α-amino acid derivatives of glycine type promote the degradation of desmoglein (corneodesmosomal protein).

### SUMMARY OF THE INVENTION

In the investigation of the molecular structure/activity relationships by an in vitro test of corneodesmosomal degradation, it has now surprisingly and unexpectedly been found that 2-oxothiazolidine-4-carboxylic acid derivatives are capable of stimulating the degradation of corneodesmosine, undoubtedly by promoting the activity of proteases (of chymotrypsin-like and trypsin-like type in particular) involved in this process.

These 2-oxothiazolidine-4-carboxylic acid derivatives thus constitute excellent active agents for promoting the desquamation of the skin and/or for stimulating epidermal renewal and thus for combating aging of the skin.

The present invention thus features novel compositions comprising at least one L-2-oxothiazolidine-4-carboxylic acid derivative (also known as “procysteine”), to promote desquamation of the skin and/or to stimulate epidermal renewal and thus to combat intrinsic aging of the skin.

Admittedly, the use of procysteine has already been the subject of many studies and patents, especially with the aim of protecting the body against various types of stress. Thus, the patents from Cornell University cover the use of procysteine for therapeutic purposes and as a glutathione inducer (U.S. Pat. Nos. 4,335,210; 4,434,158; 4,438,124 and 4,647,751).

Patents from the company Clintec (EP-501,641, EP-501,637, EP-535,390 and U.S. Pat. No. 5,214,062) also describe the use of procysteine for the therapeutic treatment of disorders, among which mention may be made of liver, heart and viral disorders.

In addition, EP-A-656,201 (patent from the company Free Radical Sciences) discloses other L-2-oxothiazolidine-4-carboxylic acid esters used as cysteine precursors and in combination with glutathione stimulators to prevent hair loss and to stimulate the growth of new hair.

Moreover, the depigmenting properties of procysteine have been described in EP-A-780,120.

EP-A-1,038,515 mentions the use of procysteine:
for depigmenting or bleaching the skin, head hair and/or other hairs,
for preventing hair loss and/or stimulating hair regrowth,
for preventing or treating photoaging and/or environmental stress, in particular on account of the free-radical-scavenging properties of L-oxothiazolidine-4-carboxylic acid,
and/or for preventing or treating greasy skin, for example in the treatment of acne.

On the other hand, the prodesquamating properties of L-2-oxothiazolidine-4-carboxylic acid and its derivatives were not known hitherto.

Thus, to date it has never been described in the prior art that L-2-oxothiazolidine-4-carboxylic acid and its derivatives according to the invention are capable of stimulating the degradation of corneodesmosine and may thus constitute excellent active agents for promoting desquamation of the skin and/or for stimulating epidermal renewal and thus combating intrinsic aging of the skin.

In addition, many skin pathologies are characterized by the production of a thickened horny layer and by abnormal desquamation, i.e., hyperkeratosis. This may occur on any anatomical region of skin and in very varied clinical contexts. Its physiopathological substratum and its cause are varied.

Examples that may be mentioned include:
xerosis (or dryness of the skin),
ichthyosis,
psoriasis,
certain benign or malignant tumoral lesions,
reactional hyperkeratosis.

Thus, L-2-oxothiazolidine-4-carboxylic acid and its derivatives according to the invention are capable of stimulating the degradation of corneodesmosine and thus constitute excellent active agents for promoting desquamation of the skin and/or for stimulating epidermal renewal and thus for treating skin pathologies characterized by the production of a thickened horny layer and by abnormal desquamation.

This invention thus features formulating at least one 2-oxothiazolidine-4-carboxylic acid compound corresponding to formula (I) below: 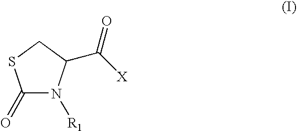
in which,
X represents an —OH radical or a radical —NHR₂, and R₁ and R₂, which may be identical or different, represent:
   a hydrogen atom,
   a linear or branched C1-C8 alkyl radical, optionally substituted with at least one radical —OR and/or one radical —COOR and/or one radical —NHR and/or one radical —SR and/or one radical —R for which R represents a hydrogen or a linear or branched C1-C4 alkyl,
   a radical COR₃ in which R₃ represents a linear or branched C1-C8 alkyl, optionally substituted with at least one radical —OR and/or one radical —COOR and/or one radical —NHR and/or one radical —SR and/or one radical —R for which R represents a hydrogen or a linear or branched C1-C4 alkyl,
   a benzenic or heterocyclic aralkyl or aryl radical optionally substituted with at least one radical —OR and/or one radical —COOR and/or one radical —NHR and/or one radical —SR and/or one radical —R for which R represents a hydrogen or a linear or branched C1-C4 alkyl, into a cosmetic composition comprising a physiologically acceptable medium, as an agent for promoting desquamation of the skin and/or for stimulating epidermal renewal and thus advantageously for combating intrinsic aging of the skin.

This invention also relates to the optical and/or geometrical isomers of the derivatives of formula (1), alone or as a mixture in all proportions, and also the physiologically acceptable salts of these derivatives.

According to the invention, the terms “linear or branched C1-C4 alkyl” and “linear or branched C1-C8 alkyl” mean acyclic radicals derived from the removal of a hydrogen atom in the linear or branched hydrocarbon molecule containing from 1 to 4, or respectively 1 to 8, carbon atoms and in particular methyl, ethyl, propyl, isopropyl, butyl, tert-butyl, pentyl, hexyl and heptyl radicals, and also the corresponding positional isomers thereof.

The expression “physiologically acceptable medium” means a medium that is compatible with the skin, mucous membranes, the nails, the scalp and the hair.

Needless to say, according to the invention, the 2-oxothiazolidine-4-carboxylic acid derivatives of formula (I) may be used alone or as a mixture in all proportions.

In the text hereinbelow, the term “2-oxothiazolidine-4-carboxylic acid derivative of formula (1)” denotes the derivatives described above, of natural or synthetic origin, totally or partially purified, or any preparation containing them.

The expression “natural origin” means a derivative extracted from natural material in which it is present. The expression “synthetic origin” means a derivative prepared by chemical synthesis or by biotechnology.

The expression “totally or partially purified” means herein that, during its synthesis or compared with its natural state (fresh or dried plant or cells), the 2-oxothiazolidine-4-carboxylic acid derivative of formula (I), in the composition of the invention, has been concentrated and/or freed, respectively, of at least some of the reaction side products derived from its synthesis or of at least some of the other constituents of the plant.

The present invention also features the use of a cosmetic composition comprising, in a physiologically acceptable medium, at least one 2-oxothiazolidine-4-carboxylic acid derivative of formula (1) as defined above, in a regime or regimen to promote desquamation of the skin and/or to stimulate epidermal renewal and thus advantageously to combat intrinsic aging of the skin.

This invention also features the use of at least one 2-oxothiazolidine-4-carboxylic acid derivative of formula (I) as defined above, for the manufacture of a pharmaceutical or dermatological composition comprising a physiologically acceptable medium, said composition being intended to promote desquamation of the skin and/or to stimulate epidermal renewal and thus advantageously to combat intrinsic aging of the skin.

This invention also features administration of at least one 2-oxothiazolidine-4-carboxylic acid derivative of formula (I) as defined above, to an individual in need of such treatment in a pharmaceutical or dermatological composition comprising a physiologically acceptable medium, said composition being intended to treat skin pathologies characterized by the production of a thickened horny layer-and by abnormal desquamation, particularly xerosis or dryness of the skin, ichthyosis, psoriasis, benign or malignant tumoral lesions, and reactional hyperkeratosis.

### DETAILED DESCRIPTION OF BEST MODE AND SPECIFIC/PRFEFERRED EMBODIMENTS OF THE INVENTION

Advantageously, the derivatives used according to the invention are those of formula (I) in which X represents an —OH radical or a radical —NHR₂, and R₁ and R₂, which may be identical or different, represent:
a hydrogen atom,
an unsubstituted linear C1-C4 alkyl radical, and advantageously a methyl radical,
a linear C1-C4 alkyl radical substituted with at least one radical —COOR and/or one radical —NHR in which R represents a hydrogen or a linear or branched C1-C4 alkyl,
a radical COR₃ in which R₃ represents a linear C1-C4 alkyl substituted with at least one radical —COOR and/or one radical —NHR in which R represents a hydrogen or a linear or branched C1-C4 alkyl,
an unsubstituted benzenic or heterocyclic aralkyl or aryl radical.

Among the derivatives of formula (I) administered according to the invention, the ones most particularly preferred are:
procysteine or L-2-oxothiazolidine-4-carboxylic acid, which corresponds to the following formula: 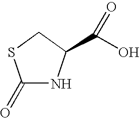
   -2-oxo-3-(L-γ-glutamyl)thiazolidine-4-carboxylic acid, which corresponds to the following formula: 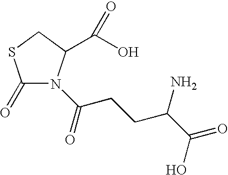
   -N-(2-oxothiazolidin-4-ylcarbonyl)glycine, which corresponds to the following formula: 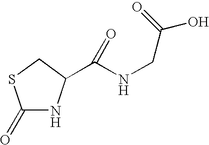
   -N-[2-oxo-3-(L-γ-glutamyl)thiazolidin-4-ylcarbonyl]glycine, which corresponds to the following formula:

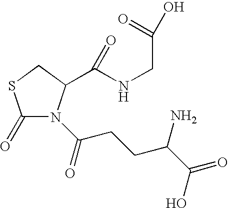

The amount of 2-oxothiazolidine-4-carboxylic acid derivative of formula (I) that may be used according to the invention obviously depends on the desired effect and must be in an amount that is effective for promoting desquamation of the skin and/or for stimulating epidermal renewal and thus for combating intrinsic aging of the skin.

By way of example, the amount of 2-oxothiazolidine-4-carboxylic acid derivative of formula (I) that may be used according to the invention may range, for example, from 0.01% to 50% and preferably from 0.1% to 10% of the total weight of the composition.

Given its good solubility in water (greater than 4%) and in ethanol (greater than 10%), procysteine affords the additional advantage of being easy to formulate at 1% in a large number of cosmetic formulations of the type such as W/O or O/W emulsions, liposomes or oleosomes, provided that the pH is maintained between 5 and 8.

The composition according to the invention may be intended for cosmetic or pharmaceutical, and particularly dermatological, application.

The composition according to the invention may be ingested, injected or applied to the skin (to any area of body skin), the hair, the nails or mucous membranes (oral, jugal, gingival, genital or conjunctival membranes).

Depending on the mode of administration, the composition according to the invention may be in any pharmaceutical form normally used, particularly in cosmetology.

One preferred composition of the invention is a cosmetic composition intended for topical application.

For a topical application to the skin, the composition which may be used according to the invention may especially be in the form of an aqueous or oily solution or of a dispersion of the lotion or serum type, of emulsions of liquid or semi-liquid consistency of the milk type, obtained by dispersing a fatty phase in an aqueous phase (O/W emulsion) or conversely (W/O emulsion), or of suspensions or emulsions of soft consistency of the aqueous or anhydrous cream or gel type, or alternatively of microcapsules or microparticles, or of vesicular dispersions of ionic and/or nonionic type.

These compositions are prepared according to the usual methods. The composition which may be used according to the invention may also be a haircare composition, and especially a shampoo, a setting lotion, a treating lotion, a styling cream or gel, a dye composition (especially for oxidation dyeing) optionally in the form of coloring shampoos, restructuring lotions for the hair, a permanent-waving composition (especially a composition for the first stage of a permanent-waving operation), a lotion or gel for preventing hair loss, an antiparasitic shampoo, etc.

The amounts of the various constituents of the compositions which may be used according to the invention are those that are conventionally used in the fields under consideration.

These compositions especially constitute cleansing, protective, treating or care creams for the face, for the hands, for the feet, for the major anatomical folds or for the body (for example day creams, night creams, make-up-removing creams, foundation creams and antisun creams), fluid foundations, make-up-removing milks, protective body milks or bodycare milks, after-sun milks, skincare lotions, gels or mousses, for instance cleansing lotions, antisun lotions, artificial tanning lotions, bath compositions, deodorant compositions comprising a bactericidal agent, aftershave gels or lotions, hair-removing creams, insect-repellent compositions, pain-relief compositions, compositions for treating certain skin diseases, for instance eczema, acne rosacea, psoriasis, lichens and severe pruritus.

The compositions which may be used according to the invention may also consist of solid preparations constituting cleansing soaps or bars.

The compositions which may be used according to the invention may also be packaged in the form of an aerosol composition also comprising a pressurized propellant.

When the composition which may be used according to the invention is an emulsion, the proportion of the fatty phase may range from 5% to 80% by weight and preferably from 5% to 50% by weight relative to the total weight of the composition. The oils, waxes, emulsifiers and co-emulsifiers used in the composition in emulsion form are chosen from those conventionally used in cosmetics. The emulsifier and co-emulsifier are present in the composition in a proportion ranging from 0.3% to 30% by weight and preferably from 0.5% to 20% by weight relative to the total weight of the composition. The emulsion may also contain lipid vesicles.

When the composition which may be used according to the invention is an oily solution or gel, the fatty phase may represent more than 90% of the total weight of the composition.

In a known manner, the cosmetic composition may also contain adjuvants that are common in cosmetics, such as hydrophilic or lipophilic gelling agents, hydrophilic or lipophilic additives, preserving agents, antioxidants, solvents, fragrances, fillers, screening agents, odor absorbers and dyestuffs. The amounts of these various adjuvants are those conventionally used in cosmetics and, for example, from 0.01% to 10% of the total weight of the composition. Depending on their nature, these adjuvants may be introduced into the fatty phase, into the aqueous phase and/or into the lipid spherules.

As oils or waxes which may be used in the invention, mention may be made of mineral oils (liquid petroleum jelly), plant oils (liquid fraction of karite butter or sunflower oil), animal oils (perhydrosqualene), synthetic oils (purcellin oil), silicone oils or waxes (cyclomethicone) and fluoro oils (perfluoropolyethers), beeswax, carnauba wax or paraffin wax. Fatty alcohols and fatty acids (stearic acid) may be added to these oils.

As emulsifiers which may be used in the invention, mention may be made, for example, of glyceryl stearate, polysorbate 60 and the mixture of PEG-6/PEG-32/glycol stearate sold under the name Tefose® 63 by the company Gattefosse.

As solvents which may be used in the invention, mention may be made of lower alcohols, especially ethanol and isopropanol, and propylene glycol.

As hydrophilic gelling agents which may be used in the invention, mention may be made of carboxyvinyl polymers (carbomer), acrylic copolymers such as acrylate/alkyl acrylate copolymers, polyacrylamides, polysaccharides such as hydroxypropylcellulose, natural gums and clays, and, as lipophilic gelling agents, mention may be made of modified clays, for instance bentones, metal salts of fatty acids, for instance aluminum stearates, and hydrophobic silica, ethylcellulose and polyethylene.

The compositions which may be used according to the invention may contain other hydrophilic active agents, for instance proteins or protein hydrolyzates, amino acids, polyols, urea, allantoin, sugars and sugar derivatives, water-soluble vitamins, plant extracts and hydroxy acids.

Lipophilic active agents which may be used include retinol (vitamin A) and its derivatives, tocopherol (vitamin E) and its derivatives, essential fatty acids, ceramides, essential oils and salicylic acid and its derivatives.

The compositions which may be used according to the invention may combine at least one 2-oxothiazolidine4-carboxylic acid derivative of formula (I) with other active agents. Among these active agents which may be mentioned, for example, are:
agents for modulating skin differentiation and/or proliferation and/or pigmentation, such as retinoic acid and its isomers, retinol and its esters, vitamin D and its derivatives, estrogens such as estradiol, kojic acid or hydroquinone;
antibacterial agents such as clindamycin phosphate or erythromycin or antibiotics of the tetracycline family;
antiparasitic agents, in particular metronidazole, crotarniton or pyrethroids;
antifungal agents, in particular compounds belonging to the imidazole family, such as econazole, ketoconazole or miconazole or their salts, polyene compounds, such as arnphotericin B, compounds of the allylamine family, such as terbinafine, or alternatively octopirox;
antiviral agents such as acyclovir;
steroidal anti-inflammatory agents, such as hydrocortisone, betamethasone valerate or clobetasol propionate, or non-steroidal anti-inflammatory agents such as, for example, ibuprofen and its salts, diclofenac and its salts, acetylsalicylic acid, acetaminophen or glycyrrhizic acid;
aensthetics such as lidocaine hydrochloride and its derivatives;
antipruriginous agents, for instance thenaldine, trimeprazine or cyproheptadine;
agents acting on the radiance of the complexion by promoting turnover and desquamation (keratolytic agents), such as α- and β-hydroxycarboxylic acids or β-keto carboxylic acids, their salts, amides or esters and more particularly hydroxy acids such as glycolic acid, lactic acid, salicylic acid, citric acid and fruit acids in general, and 5-n-octanoylsalicylic acid;
free-radical scavengers, such as a-tocopherol or its esters, superoxide dismutases, certain metal chelating agents or ascorbic acid and its esters;
antiseborrheic agents such as progesterone;
antidandruff agents, for instance octopirox or zinc pyrithione;
antiacne agents, for instance, retinoic acid or benzoyl peroxide.

Other compounds may also be added to the above list, namely, for example Diazoxide, Spiroxazone, phospholipids, for instance lecithin, linoleic acid, linolenic acid, salicylic acid and its derivatives described in FR-2,581,542, for instance salicylic acid derivatives bearing an alkanoyl radical containing from 2 to 12 carbon atoms in position 5 of the benzene ring, hydroxycarboxylic acids or keto carboxylic acids and their esters, lactones and their corresponding salts, anthralin, carotenoids, eicosatetraenoic acid and eicosatrienoic acid or their esters and amides.

Thus, according to one particular embodiment, the composition according to the invention also comprises at least one agent chosen from antibacterial agents, antiparasitic agents, antifungal agents, antiviral agents, anti-inflammatory agents, antipruriginous agents, anesthetics, keratolytic agents, free-radical scavengers, antiseborrheic agents, antidandruff agents, antiacne agents and/or agents for modulating skin differentiation and/or proliferation and/or pigmentation, and extracts of plant, marine or bacterial origin, or mixtures thereof.

It may also be envisaged that the composition used according to the invention comprising at least one derivative of formula (I) as defined above is in liposomal form, as described especially in WO 94/22468 filed on Oct. 13, 1994 by the company Anti Cancer Inc.

According to another aspect, a subject of the invention is a composition comprising at least a combination of at least one 2-oxothiazolidine-4-carboxylic acid derivative of formula (I) and of at least one other prodesquamating agent.

The other prodesquamating agents are prodesquamating agents which are known for their moisturizing properties and/or which act on the radiance of the complexion by promoting turnover and desquamation (keratolytic agents).

The other prodesquamating agents known for their moisturizing properties are chosen from glycerol and urea and derivatives thereof, pyrrolidonecarboxylic acid, and ammonium salts of lactic acid.

The other prodesquamating agents which act on the radiance of the complexion by promoting turnover and desquamation (keratolytic agents) are chosen from hydroxy acids, in particular—and -hydroxycarboxylic acids or -keto carboxylic acids, and their salts, amides or esters, and more particularly hydroxy acids such as glycolic acid, lactic acid, salicylic acid, citric acid and fruit acids in general, and 5-n-octanoylsalicylic acid.

One embodiment of the invention thus features a nontherapeutic method for treating the skin which is intended for promoting desquamation of the skin and/or for stimulating epidermal renewal, wherein a cosmetic composition comprising at least one 2-oxothiazolidine-4-carboxylic acid derivative of formula (I) as defined above is topically applied to the skin.

Another embodiment of the invention is a nontherapeutic method for combating intrinsic aging of the skin, wherein a cosmetic composition comprising at least one 2-oxothiazolidine-4-carboxylic acid derivative of formula (I) as defined above is topically applied to the skin.

Another embodiment of the invention is a method for promoting desquamation of the skin and/or for stimulating epidermal renewal and thus for combating aging of the skin in an individual displaying abnormally low skin desquamation and/or abnormally low epidermal renewal, comprising the topical application to the skin of an effective amount of at least one 2-oxothiazolidine-4-carboxylic acid derivative of formula (I) as defined above.

This invention also features a method for promoting desquamation of the skin and/or for stimulating epidermal renewal in an individual displaying production of a thickened horny layer and abnormal desquamation, comprising the topical application to the skin of an effective amount of at least one 2-oxothiazolidine-4-carboxylic acid derivative of formula (I) as defined above.

In order to further illustrate the present invention and the advantages thereof, the following specific examples are given, it being understood that same are intended only as illustrative and in nowise limitative.

### EXAMPLE 1

Method for Evaluating Desquamation by Measuring the Degradation of Corneodesmosines

The ability of 2-oxothiazolidine-4-carboxylic acid derivatives of formula (I) according to the invention to promote desquamation by degradation of corneodesmosines is studied in this example.

Corneodesmosine is one of the major markers of desquamation of the corneodesmosome. It is studied by immunoblotting after separation by electrophoresis and transfer onto a membrane. After a specific labeling with monoclonal antibody G36-19, it is revealed by chemiluminescence.

The mouse monoclonal antibody G36-19 is specific for comeodesmosine; it forms part of a series of antibodies directed against epidermal differentiation antigens, produced after immunizing a mouse with a homogenate of human plantar horny layer, and then characterized (Serre G. et al., J. Invest. Dermatol. 1991; 97(6):1061-72).

Varnish-stripping operations are carried out on the lower legs of volunteers (modification of the procedure by Lundstrom A. and Egelrud T., Acta Dern. Venereol. (Stockholm) 71, 471-474, 1991). The nylon-varnish strips associated with the corneocytes are immersed in 1 ml/cm² of acetone in order to detach the corneocytes. The mixture is filtered and then rinsed three times with the same volume of acetone in order to remove all trace of varnish. Finally, the mixture is dried under vacuum: acetonic powders of stratum corneum are thus obtained.

The acetonic powders are divided into 1 mg aliquots. 100 μl of the aqueous solutions containing 2% of active agent adjusted to pH 8.0 are added. Controls without active agent are prepared under the same conditions. Two incubation times are studied: t=0 and t=17 h. In the latter case, the incubation takes place at 30° C. with stirring.

After incubation, the mixtures are centrifuged for 10 minutes at 10 000 ×g. The supernatant is removed and replaced with 100 μL of 0.0625 M Tris/HCl pH 6.8 Laemmli buffer, 2% SDS, 200 mM DTT, 10% glycerol, which allows the proteins to be extracted. The mixture is boiled for 10 minutes at 100° C. and then ground in a Potter mill. The mixture is centrifuged for 10 minutes at 10 000 ×g and the supernatant is then collected. It contains the corneodesmosomal proteins.

The total proteins are assayed according to the Bradford method (Biorad kit). This allows an adjustment to 0.6 mg/ml of the samples and a real comparison of the treatments.

The samples and also a Rainbow (Amersham Pharmacia Biotech) low molecular weight standard at 1/3 are separated by electrophoresis on gel containing 12% acrylamide for 30 minutes at 100 V and then for 1 hour at 200 V. After the electrophoresis, the proteins are transferred onto an Immobilon-P membrane (Millipore) for 3 hours at 60 V. The membrane is then incubated for twice 15 minutes in TBS-TL buffer: 25 mM Tris, 0.15M NaCl pH 7.2, 0.05% Tween 20, 0.5% skimmed milk powder, in order to block the non-specific sites. Incubation with the antibody G36-19 at 1/12 500 is performed overnight at 4° C. After two rinses of 5 minutes in TBS-TL buffer, the membrane is incubated with a goat anti-mouse IG(H+L) antibody peroxidase conjugate (Biorad) at 1/4000 for 1 hour 30 minutes at ambient temperature. After several rinses of 5 minutes in TBS-TL buffer and then TBS buffer (without milk or Tween), the membrane is incubated for 1 minute in 10 ml of ECL reagent (Amersham Pharmacia Biotech). The chemiluminescence of the corneodesmosine bands is measured with the FluorS Multimager (Biorad). The 33 and 46 kD bands are quantified with the Quantity-one software (Biorad).

The results of this study are summarized in the table below:

TABLE
Effect of L-2-oxothiazolidine-4-carboxylic acid
(procysteine) on corneodesmosine degradation

| | Percentage increase in |
|---|---|
| Test Molecule | corneodesmosine degradation |
| Control | 0% |
| Procysteine | 77% |

The Control corresponds to a control prepared with the dissolution buffer without active agent under the same conditions of the test. This control takes into account the natural degradation of the corneodesmosines that takes place during the incubation.

It emerges clearly that procysteine promotes the degradation of corneodesmosines.

### EXAMPLE 2

Compositions:

Prodesquamating cream for the face:

| | |
|---|---|
| L-2-Oxothiazolidine-4-carboxylic acid | 2.00% |
| Sodium stearate | 3.00% |
| Liquid petroleum jelly | 6.00% |
| Alkylparaben | 0.05% |
| Potassium sorbate | 10.00% |
| Stearyl alcohol | 1.00% |
| Fragrance | 1.00% |
| Water qs | 100.00% |

Prodesquamating cream for the body:

| | |
|---|---|
| L-2-Oxothiazolidine-4-carboxylic acid | 5.0% |
| Jojoba oil | 13.0% |
| Sipol wax | 6.0% |
| Isopropyl palmitate | 2.0% |
| Glycerol | 15.0% |
| Alkylparaben | 0.5% |
| Fragrance | 1.0% |
| Water qs | 100.0% |

Prodesquamating care cream:

| | |
|---|---|
| L-2-Oxothiazolidine-4-carboxylic acid | 1% |
| Oxyethylenated polyethylene glycol 50 | 3% |
| Mono-diglyceryl stearate | 3% |
| Liquid petroleum jelly | 24% |
| Cetyl alcohol | 5% |
| Water qs | 100% |

Desquamating care cream for the body:

| | |
|---|---|
| L-2-Oxothiazolidine-4-carboxylic acid | 0.5% |
| Sipol wax | 6.0% |
| Glyceryl monostearate | 1.5% |
| Sodium stearate | 0.8% |
| Liquid petroleum jelly | 6.0% |
| Isopropyl palmitate | 2.0% |
| Glycerol | 15.0% |
| Fragrance | 0.3% |
| Water qs | 100.0% |

Prodesquamating care cream:

| | |
|---|---|
| L-2-Oxothiazolidine-4-carboxylic acid | 0.50% |
| Jojoba oil | 13.00% |
| Alkylparaben | 0.05% |
| Potassium sorbate | 0.30% |
| Cyclopentadimethylsiloxane | 10.00% |
| Stearyl alcohol | 1.00% |
| Stearic acid | 4.00% |
| Polyethylene glycol stearate | 3.00% |
| Vitamin E | 1.00% |
| Glycerol | 3.00% |
| Water qs | 100.00% |

Each patent, patent application and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A method for promoting desquamation of the skin in an individual afflicted with xerosis or dryness of the skin, comprising administering to said individual, a thus effective amount of at least one 2-oxothiazolidine-4-carboxylic acid compound having the structural formula (I): 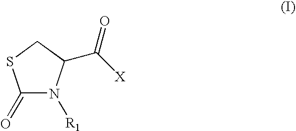
in which X is —OH or —NHR₂; and R₁ and R₂, which may be identical or different, are each a hydrogen atom, a linear or branched C₁-C₈ alkyl radical, optionally substituted with at least one radical —OR and/or one radical —COOR and/or one radical —NHR and/or one radical —SR and/or one radical —R in which R is a hydrogen atom or a linear or branched C₁-C₄ alkyl, a radical COR₃ in which R₃ is a linear or branched C₁-C₈ alkyl radical, optionally substituted with at least one radical —OR and/or one radical —COOR and/or one radical —NHR and/or one radical —SR and/or one radical —R in which R is a hydrogen atom or a linear or branched C₁-C₄ alkyl radical, a benzenic or heterocyclic aralkyl or aryl radical optionally substituted with at least one radical —OR and/or one radical —COOR and/or one radical —NHR and/or one radical —SR and/or one radical —R in which R is a hydrogen atom or a linear or branched C₁-C₄ alkyl radical, formulated into a physiologically acceptable medium therefor.

2. The method as defined by claim 1, wherein in formula (I), X is —OH or —NHR₂; and R₁ and R₂, which may be identical or different, are each a hydrogen atom, an unsubstituted linear C₁-C₄ alkyl radical, a linear C₁-C₄ alkyl radical substituted with at least one radical —COOR and/or one radical —NHR in which R is a hydrogen atom or a linear or branched C₁-C₄ alkyl, a radical COR₃ in which R₃ is a linear C₁-C₄ alkyl radical substituted with at least one radical —COOR and/or one radical —NHR in which R is a hydrogen atom or a linear or branched C₁-C₄ alkyl radical, or an unsubstituted benzenic or heterocyclic aralkyl or aryl radical.

3. The method as defined by claim 1, said at least one 2-oxothiazolidine-2-carboxylic acid compound of formula (I) comprising procysteine or L-2-oxo-4-thiazolidinecarboxylic acid; N-(2-oxothiazolidin-4-ylcarbonyl)glycine; 2-oxo-3-(L-glutamyl)thiazolidine-4-carboxylic acid; or N-[2-oxo-3-(L--glutamyl)thiazolidin-4-ylcarbonyl]glycine.

4. The method as defined by claim 1, further comprising coadministering to said individual, an effective amount of at least one other active agent selected from the group consisting of retinoic acid; retinol and its esters; vitamin D; extradiol, kojic acid, hydroquinone, clindamycin phosphate, erythromycin, a tetracycline antibiotic, metronidazole, crotamiton, a pyrethroid; econazole, ketoconazole and miconazole and their salts; amphotericin B, terbinafine, octopirox, acyclovir, hydrocortisone, betamethasone valerate, clobetasol propionate; ibuprofen and its salts; diclofenac and its salts; acetylsalicylic acid, acetaminophen, glycerrhizic acid, lidocaine hydrochloride, thenaldine, trimeprazine, cyproheptadine; α-hydroxycarboxylic acids, β-hydroxycarboxylic acids and β-keto carboxylic acids and their salts, amides and esters; α-tocopherol and its esters; superoxide dismutases; ascorbic acid and its esters; progesterone, zinc pyrithione, benzoyl peroxide, diazoxide, spiroxazone, lecithin, linoleic acid, linolenic acid, anthralin, a carotenoid; eicosatetraenoic acid and eicosatuinoic acid and their esters and amides; and mixtures thereof.

5. The method as defined by claim 1, further comprising coadministering to said individual with said at least one compound of formula (I), an effective amount of at least one other pro-desquamating agent selected from the group consisting of: hydroxy acids and their salts, amides and esters; glycerol; urea; pyrrolidonecarboxylic acid; and ammonium salts of lactic acid.

6. The method as defined by claim 5, said at least one other pro-desquamating agent being selected from the group consisting of glycerol, urea, pyrrolidonecarboxylic acid, and the ammonium salts of lactic acid.

7. The method as defined by claim 5, said at least one other pro-desquamating agent comprising a hydroxy acid, or salt, amide or ester thereof.

8. The method as defined by claim 7, said hydroxy acid, or salt, amide or ester thereof comprising an α- or β-hydroxycarboxylic acid, a β-keto carboxylic acid, or salt, amide or ester thereof.

9. The method as defined by claim 8, said hydroxy acid comprising glycolic acid, lactic acid, salicylic acid, citric acid, a fruit acid or 5-n-octanoylsalicylic acid.

10. The method as defined by claim 1, comprising topically applying said at least one 2-oxothiazolidine-4-carboxylic acid compound of formula (I) onto the skin of said individual.

11. The method as defined by claim 1, comprising administering 0.01% to 50% by weight of said at least one 2-oxothiazolidine-4-carboxylic acid compound formulated into said physiologically acceptable medium therefor.

12. The method as defined by claim 1, comprising administering 0.1% to 10% by weight of said at least one 2-oxothiazolidine-4-carboxylic acid compound formulated into said physiologically acceptable medium therefor.

13. The method as defined by claim 10, wherein said at least one of 2-oxothiazolidine-4-carboxylic acid compound of formula (I) is formulated as an aqueous or oily solution, lotion, serum, emulsion, milk, suspension, cream, gel, microcapsules, microparticles, mousse, solid, aerosol, permanent wave, or shampoo.

14. The method as defined in claim 1, wherein X is —OH.

15. The method as defined in claim 1, wherein R₁ is hydrogen.

16. The method as defined in claim 1, wherein X is —OH and R₁ is hydrogen.
